(19) United States
(12) Patent Application Publication
(10) Pub. No.: US 2008/0208050 A1
(43) Pub. Date: Aug. 28, 2008

(54) ULTRASONIC IMAGING APPARATUS AND ULTRASONIC IMAGE ACQUISITION METHOD

(75) Inventor: Kenichi ICHIOKA, Nasushiobara-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS CORPORATION, Otawara-Shi (JP)

(21) Appl. No.: 12/037,568

(22) Filed: Feb. 26, 2008

(30) Foreign Application Priority Data
Feb. 27, 2007 (JP) 2007-046625

(51) Int. Cl. A61B 8/00

(52) U.S. Cl. 600/437; 382/128

(57) ABSTRACT

Ultrasonic image acquisition method comprising; calculating value for remaining parameter, from inputs of any values for three of four parameters, based on correlation information on four types of parameters; creating image that represents correlation graphically based on values for two of entered three parameters and obtained parameter, to create a setting screen on which display part variably displays parameters; displaying setting screen on display part; repeating calculation and setting-screen-creation by defining entered values for three parameters, as values for altered parameter and two non-graphically-represented parameters, when instruction to alter value for one of two graphically represented parameters is received through input part; and irradiating ultrasound beam to object based on entered values for three parameters and obtained value for remaining parameter when determination of values for each parameter are entered, so as to generate 3D dynamic image based on data obtained by receiving ultrasonic echo from object.

### BACKGROUND OF THE INVENTION

1. Field of the Invention

The present invention relates to an ultrasonic imaging apparatus for acquiring 3D ultrasonic images of an object and generating a 3D dynamic image (4D image) by scanning ultrasound beam from inside the subject. In particular, it relates to a user interface for setting parameters to be used to generate a 3D dynamic image by using such an ultrasonic imaging apparatus.

2. Description of the Related Art

The ultrasonic diagnostic equipment irradiates an ultrasound beam to the object through an ultrasonic probe and receives the reflected wave, to convert into luminance the amplitude of the reflected wave from depth and to acquire a 2D cross-sectional image of the object. The 2D cross-sectional imaging acquired at this time is called the B-mode image. Moreover, by moving the position of the scanned surface in the orthogonal direction to the surface to scan adjacent multiple cross-sectional surfaces, it is possible to obtain a 3D ultrasonic image (3D image) in the 3D-scanned volume (i.e. a range of 3D space that the 3D figure occupies, in which adjacent 2D cross-sectional images are configured as one 3D figure). Furthermore, by repeating the 3D scans with ultrasound beams to the same volume and displaying it in series, it is possible to obtain 3D ultrasonic images of the object in the volume in real time as a dynamic image. Herein, real time refers to displaying images at substantially the same time as when the ultrasonic images are obtained. The real time dynamic image of such 3D ultrasonic images is called a 3D dynamic image. This 3D dynamic image may also be called a 4D ultrasonic image. Herein, the moving direction of the ultrasound beam when creating 2D cross-sectional imaging is referred to as the “slice direction” and the direction orthogonal to the slice direction is referred to as the “volume direction.”

Four types of parameters are required for creating a 3D dynamic image. The first parameter is a parameter to create a 2D cross-sectional image. Creating a 2D cross-sectional image requires an irradiation range (e.g., an oscillating angle) in the slice direction. The slice direction is indicated by a start point and an end point to displace the ultrasound beam in the scanning direction to determine the range for creating a cross-sectional surface. With larger parameters, 2D cross-sectional images with broader range can be created. That is, the scanning range of the 3D ultrasonic image in the scanning direction is broadened.

The second parameter is a parameter for creating a 3D ultrasonic image. Creating a 3D ultrasonic image requires an irradiation range (the “scan range”) in the volume direction. The volume direction is indicated by a start point and an end point to displace the cross-sectional surface in the volume direction to determine the range that is to be the volume. This parameter unit varies depending on the scanning method, including the moving distance and angle. The larger this parameter, the broader the scanning range of the 3D ultrasonic image becomes in the volume direction.

The third parameter is image density (roughness). With higher density, a clearer image is generated. On the other hand, with lower density, a less clear image is generated.

The fourth parameter is volume rate. The volume rate refers to the number of volumes that can be created per unit time (e.g., per second). With a faster (higher) volume rate, the number of 3D ultrasonic images displayed per second increases, which leads to smooth temporal movement of the 3D dynamic image. With a slower (lower) volume rate, the temporal movement of the 3D dynamic image becomes rougher.

By setting the above mentioned four types of parameters, one can determine a state of the 3D dynamic image such as a broad range, a clear image, and a smoothly moving image.

The processing capability of the ultrasonic diagnostic equipment is limited. Therefore, within the processing capability, the four types of parameters explained above are correlated to achieve real-time display. In short, as the oscillating angle increases, it requires more processing capability of the ultrasonic diagnostic equipment, therefore making it necessary to decrease the values for other parameters. As the scan range increases, it requires more processing capability of the ultrasonic diagnostic equipment, therefore making it necessary to decrease the values for other parameters. In addition, as the image density increases, it requires more processing capability of the ultrasonic diagnostic equipment, therefore making it necessary to decrease the values for other parameters. Furthermore, as the volume rate increases, it requires more processing capability of the ultrasonic diagnostic equipment, therefore making it necessary to decrease the values for other parameters. Accordingly, said four types of parameters are in a trade-off relationship in which, when the processing capability of the ultrasonic diagnostic equipment is specified at a certain level, if one of the parameters increases, one or more of the other parameters must be decreased. Specifically, when the oscillating angle and the scan range are fixed and the density is altered to a larger value, it is necessary to alter the volume rate to be a smaller value for the real-time display. Such a trade-off relationship can be established between any of the parameters.

In conventional ultrasonic imaging apparatuses, users or operators set each parameter using user interfaces on which the correlation of parameters are not reflected, such as in interfaces displaying the value for each parameter only. These ultrasonic imaging apparatuses have displayed only the values for respective parameters set by the user or operator on the user interface.

(For example, see Japanese Unexamined Patent Application Publication 2006-026256.)

However, if each parameter is set independently, the trade-off relationship is reflected automatically. Therefore, the resultant volume rate may become lowered to a level beyond the user or operator's intention, and thus it requires the user or operator to have experience-based skills. As such, because it requires such specialized skills, it has been difficult for users or operators with little experience to create a 3D dynamic image.

### SUMMARY OF THE INVENTION

The present invention has been developed by taking such circumstances into account. The present invention intends to provide an ultrasonic diagnostic equipment that displays the correlation of four parameters used for generating a 3D dynamic image based on each independently set value. The four parameters include the irradiation range of an ultrasound beam in the slice direction, scan range, volume rate, and image density age to be visually recognized. To alter the values for parameters thereafter, the setting screen is utilized that represents the correlations between the parameters, allowing each parameter to be set easily.

The first aspect of the present invention is an ultrasonic imaging apparatus comprising: a user interface having a display part and an input part to enter values for three among four parameters including an irradiation range of an ultrasound beam in a slice direction, an irradiation range of an ultrasound beam orthogonal to said slice direction, an image density, and the number of 3D image displays per unit time; a parameter-calculator configured to calculate the value for the remaining parameter based on said values for three parameters and correlation information that represents the correlations between said four parameters stored in advance in a memory; a graphic-creating part configured to execute: creating an image that represents the correlations graphically based on the values for two of said three entered parameters and said obtained parameter; creating a setting screen to instruct said display part to variably display both parameters and, in the case of receiving an instruction to alter the value for one of said two graphically expressed parameters, instructing said parameter-calculator to determine the other value based on said correlation information, so as to recreate said graphically expressed image; a display controller configured to instruct the display part to display said setting screen; and an ultrasonic image generator configured to generate a 3D dynamic image based on the data obtained by receiving an input to determine the value for said parameter, irradiating an ultrasound beam to an object based on said values for four parameters, and receiving an ultrasonic echo.

The second aspect of the present invention is an ultrasonic image acquisition method comprising; calculating the value for the remaining parameter, from inputs of any values for three of four parameters, based on the correlation information on said four types of parameters stored in advance in the memory, the four parameters including: an irradiation range of an ultrasound beam in a slice direction, an irradiation range of an ultrasound beam orthogonal to said slice direction, an image density, and the number of 3D image displays per unit time; creating an image that represents the correlation graphically based on values for two of said entered three parameters and said obtained parameter, to create a setting screen on which said display part variably displays the parameters; displaying said setting screen on said display part; repeating said calculation and said setting-screen-creation by defining said entered values for three parameters, as the values for the altered parameter and two non-graphically-represented parameters, when an instruction to alter the value for one of said two graphically represented parameters is received through the input part; and irradiating an ultrasound beam to an object based on said entered values for three parameters and said obtained value for the remaining parameter when determination of values for each parameter are entered, so as to generate a 3D dynamic image based on the data obtained by receiving an ultrasonic echo from the object.

According to the ultrasonic imaging apparatus in the first aspect and the ultrasonic image acquisition method in the second aspect, when creating a 3D dynamic image, it is possible to graphically represent four parameters in a trade-off relationship. It is also possible to adjust said four parameters by using the setting screen for the graphically displayed parameters. It allows for the visual recognition of the setting conditions of the four parameters and the easy setting of the four parameters required to create the 3D dynamic image.

### BRIEF DESCRIPTION OF THE DRAWINGS

FIG. 1 is a block diagram of the ultrasonic imaging apparatus according to the first and second embodiments.

FIG. 2 is a diagram that shows one example of the setting screen of graphically displayed parameters.

FIG. 3 is a diagram that shows one example of the setting screen of graphically displayed parameters altered by changing the parameters.

FIG. 4 is a flow chart of parameter settings and 3D dynamic image creation of the ultrasonic diagnostic equipment according to the first embodiment.

### DETAILED DESCRIPTION OF THE EMBODIMENTS

### First Embodiment

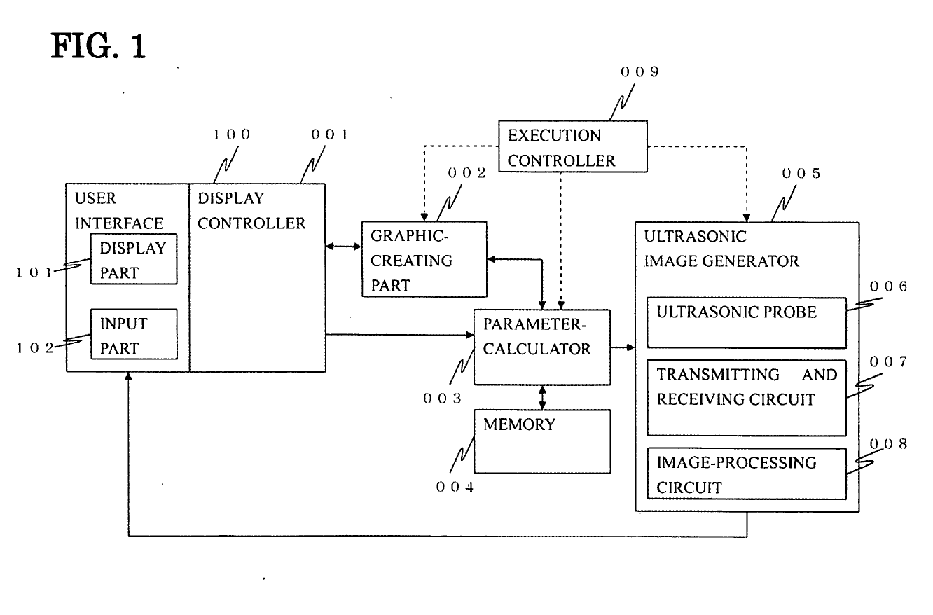

The ultrasonic diagnostic equipment according to the first embodiment of the present invention is explained below. FIG. 1 is a block diagram that represents the functions of the ultrasonic imaging apparatus according to the present invention. The display controller 001, the graphic-creating part 002, the parameter-calculator 003, the ultrasonic image controller 005, and the execution controller 009 are implemented respectively by CPU and programs that specify performance of CPU.

The memory 004 is a data storage media such as a hard disk or nonvolatile memory. The memory 004 preliminarily stores a corresponding table that represents by numerical values the correlation of four parameters required for creating a 3D dynamic image, including the irradiation range of ultrasound beam in the slice direction (the “oscillating angle”), the irradiation range of ultrasound beams in the direction orthogonal to the slice direction (the “scan range”), the image density, and the number of 3D ultrasonic image displays per unit time (the “number of displays per second (vps: Volume Per Second)”. This parameter is also referred to as “volume rate” below). These correlations are each in a trade-off relationship. Herein the number of 3D ultrasonic image displays per unit time refers to the number of frames displayed on the screen. When multiple images are displayed in one frame, one image considered to consist of the displayed multiple images refers to the number of images displayed per unit time.

In addition, when two of the four parameters are fixed values, the abovementioned correlations have a trade-off relationship for the remaining two parameters, wherein as one increases, another decreases. Furthermore, each parameter has its own multi-step value. In the present embodiment, the oscillating angle is set to a value in which the angle gradually increases from the first angle to the eighth angle (unit: degrees). The scan range is set to a value in which the distance gradually increases from the first range to the eighth range (unit: mm). The image density is set to a value in which the density gradually increases from the first density to the eighth density. Moreover, the volume rate is set to a value in which the volume number to be displayed per second gradually increases from the first rate to the eighth rate (unit: vps).

The operator uses the user interface 100 having the display part 101 and the input part 102 to instruct the parameter-calculator 003 to enter values for three of the four parameters (oscillating angle, scan range, image density, and volume rate). The input part 102 may be a keyboard, a track ball, or a touch panel. The reason for entering three parameters herein is to determine all the parameters individually.

The parameter-calculator 003 receives the values for three parameters entered by the operator from the display controller 001. Hereinafter, the oscillating angle, scan range, image density, and volume rate are represented as w, x, y, and z, respectively. The explanation below is for cases in which the oscillating angle (w), scan range (x), and image density (y) are entered as (w, x, y)=(w₁, x₁, y₁), respectively.

The parameter-calculator 003 determines the value of the remaining one parameter from the received three parameters with reference to the corresponding table stored in the memory 004. Herein, the volume rate value is determined based on the values of the oscillating angle, scan range, and image density. That is, (w₁, x₁, y₁) is used to determine the z value z₁. The parameter-calculator 003 sends the entered values for three parameters and the determined values for parameter (w₁, x₁, y₁, z₁) to the graphic-creating part 002.

To alter a parameter that has already been entered, the operator specifies two of the four parameters to be fixed values and further enters the values for either one of the remaining two parameters to be altered. For example, this input alters the graphic shape by dragging the side and the top of the graphic (e.g., a square) that represents the correlation created by the graphic-creating part 002 and displayed on the display part 101 as mentioned later by using an input part 120 such as a track ball or touch panel. Hereby the value for the parameter corresponding to the length of each altered side will be entered.

The parameter-calculator 003 receives the values for the two parameters that the operator has entered as fixed values and the value for the parameter to be altered from the display controller 001. The parameter-calculator 003 takes the two fixed values and the altered value as the entered values for three parameters and refers to the corresponding table again to obtain the value for the remaining parameter. Here explains cases in which the oscillating angle and the image density are specified as the parameters to be fixed. After either the scan range value or the volume rate value has been altered, the parameter-calculator 003 refers to the corresponding table to obtain the remaining value again. That is, when w₁ and y₁ in (w, x, y, z)=(w₁, x₁, y₁, z₁) are determined to be fixed values and the scan range value is entered to alter x₂, the volume rate z₂ is determined by utilizing w₁, x₂, and y₁. Herein the scan range and the volume rate are in a trade-off relationship. Therefore, when x₂ is altered to be a value smaller than x₁, for example, the parameter-calculator 003 obtains z₂ as a value larger than z₁. The parameter-calculator 003 then sends the determined value for the parameter and the values for the other three parameters (w, x, y, z)=(w₁, x₂, y₁, z₂) to the graphic-creating part 002.

In addition, when the display controller 001 enters an instruction of determination, the parameter-calculator 003 sends the one determined value for the parameter to the ultrasonic image generator 005.

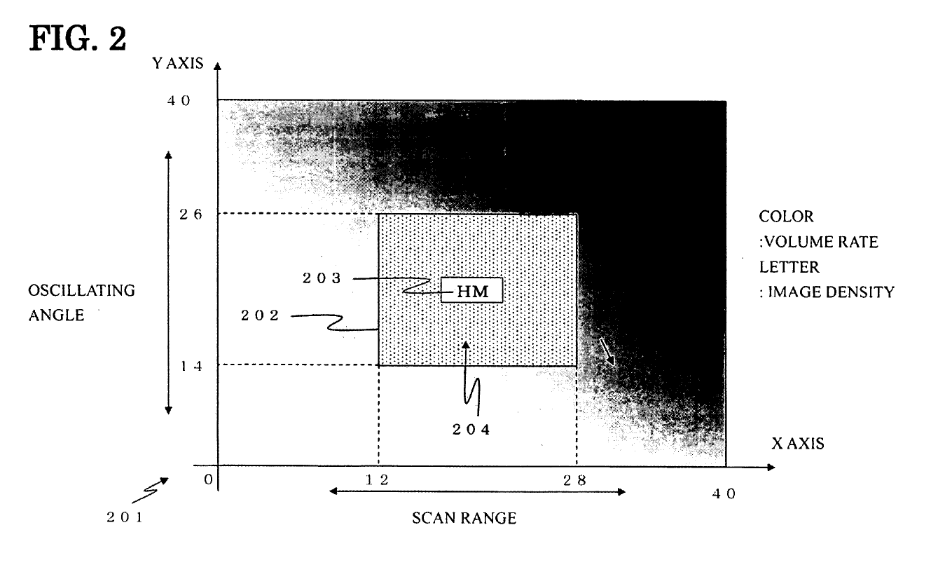

The graphic-creating part 002 receives the values for four parameters from the parameter-calculator 003. FIG. 2 shows a screen to be used for setting the parameters that are created by the graphic-creating part 002 and displayed graphically on the display part 101 (the “setting screen”). In the setting screen shown on the screen in FIG. 2, as mentioned later, the X axis represents the oscillating angle, the Y axis represents the scan range, the letter indicated in the center of the setting screen represents the image density, and the color of the square in the center of setting screen represents the volume rate.

The graphic-creating part 002 has a corresponding table of colors and letters that corresponds in advance to each level of each respective parameter. For example, it has a corresponding table in which the letters LL, ML, HL, LM, HM, LH, MH, and HH are assigned corresponding to the eight levels of image density from the lowest to the highest, respectively. In addition, the graphic-creating part 002 has a corresponding table in which the colors white, ultra-light gray, light gray, gray, dark gray, ultra-dark gray, and black are assigned corresponding to the eight levels of volume rate. In the present embodiment, the color is represented by gradation from white to black. However, it is not particularly limited to these colors. For example, gradations from red to blue or the like may be used. In the present embodiment, although the alphabet is used as letters, other letters such as katakana or numbers may be used instead. Furthermore, the user can assign which parameters are to be represented by letter or color.

In addition, the graphic-creating part 002 has a coordinate system in a size that can be displayed on the display part 101 that represents two values to generate a graphic representing the correlation depending on the values. The graphic that represents the correlation depending on the values is one example of the “image that represents the value for the parameter graphically” in the present invention. Herein, the graphic-creating part 002 has a 2D coordinate system 201 with an X axis (longitudinal axis) and a Y axis (horizontal axis) as shown in FIG. 2. The X and Y values in this coordinate system are standardized. In this case, the maximum value for both the X axis and Y axis is assumed to be 40. The graphic-creating part 002 then has a corresponding list to convert each level of the parameters to the standardized value. For example, when there are eight levels, the graphic-creating part 002 allocates the eight levels to 4, 8, 12, 16, 20, 24, 28, and 32, respectively for normalization. In the description below, the standardized values are referred to. In addition, the user can set which parameters are to correspond to the X axis and Y axis. In the present embodiment, a square is employed as a graphic to represent the correlation depending on the values for parameters. However, it may also be other graphics. For example, it may be a cube that represents the correlation of three parameters. In the following, we explain a case that employs a square that represents two parameters as a graphic to represent the correlation depending on the values for parameters. This graphic is called a correlated graphic below.

The operator enters allocation of the four parameters respectively to correlated graphic, color, or letter. The graphic-creating part 002 allocates the obtained four parameters based on the allocation of each parameter entered by the operator. In the present embodiment, as shown in FIG. 2, the graphic-creating part 002 allocates the oscillating angle to the Y-axis direction of the coordinate system 201 of the correlated graphic. In addition, the graphic-creating part 002 allocates the scan range to the X-axis direction of the coordinate system 201 of the correlated graphic. Furthermore, the graphic-creating part 002 allocates the image density to the letter. Moreover, the graphic-creating part 002 allocates the volume rate to the color. Herein, there are no specific limitations in parameter allocation. Any parameter can be displayed in any display method. In the present embodiment, the user or operator allocates the method for representing each parameter based on the allocation instructions from the user or operator. However, the graphic-creating part 002 may automatically conduct allocation without receiving such instruction.

The graphic-creating part 002 receives the values for four parameters (w, z, y, z) from the parameter-calculator 003. In the following, we consider a case in which the graphic-creating part 002 receives (w, x, y, z)=(w₁, x₁, y₁, z₁) as the values for the four parameters.

Based on the values for two parameters allocated to the correlated graphic, the graphic-creating part 002 represents one parameter as the length in the X-axis direction and another parameter as the length in the Y-axis direction. In the present embodiment, the length is represented in such a way that the center of both lengths comes to the center of the X axis and Y axis of the coordinate system 201 included in the graphic-creating part 002. In the present embodiment, the coordinate system 201 ranges from 0 to 40. For example, when the scan range value x₁ allocated in the X-axis direction takes the fourth level value, as the fourth level is standardized to be 16, it is represented by a length of 12 to 28 in the X-axis direction. When the oscillating angle value w₁ allocated in the Y-axis direction takes the third level value, as the third level is allocated to 12, it is represented by a length of 14 to 26 in the Y-axis direction. As shown in FIG. 2, the graphic-creating part 002 creates a square 202 framed by the minimum and maximum values of the length of the scan range in the X-axis direction and the minimum and maximum values of the length of the oscillating angle in the Y-axis direction to create a correlated graphic.

As shown in FIG. 2, the graphic-creating part 002 inserts the letter 203 corresponding to the values for parameters assigned to the letter into the created square 202. For example, when the image density value y₁, which is a parameter assigned to the letter, takes the fifth level value, the graphic-creating part 002 obtains the letter 203 HM corresponding to the fifth level by referring to the above-mentioned corresponding table and inserts HM (letter 203) into the square.

The graphic-creating part 002 fills in the square 202 created as shown in FIG. 2 with the color 204 corresponding to the values for parameters assigned to the color. For example, when the volume rate value z₁, which is a parameter assigned to the color, takes the third value, the graphic-creating part 002 obtains light gray, which is the color 204 corresponding to the third level, by referring to the abovementioned corresponding table and fills in the square 202 with light gray (color 204).

The graphic-creating part 002 creates a setting screen based on the correlated graphics, letters, and colors corresponding to the values for four parameters as mentioned above. Herein, the setting screen refers to a screen to set each value for the parameter while the user or operator confirms the displayed graphic, color, and letter. The graphic-creating part 002 outputs the created setting screen to the display controller 001.

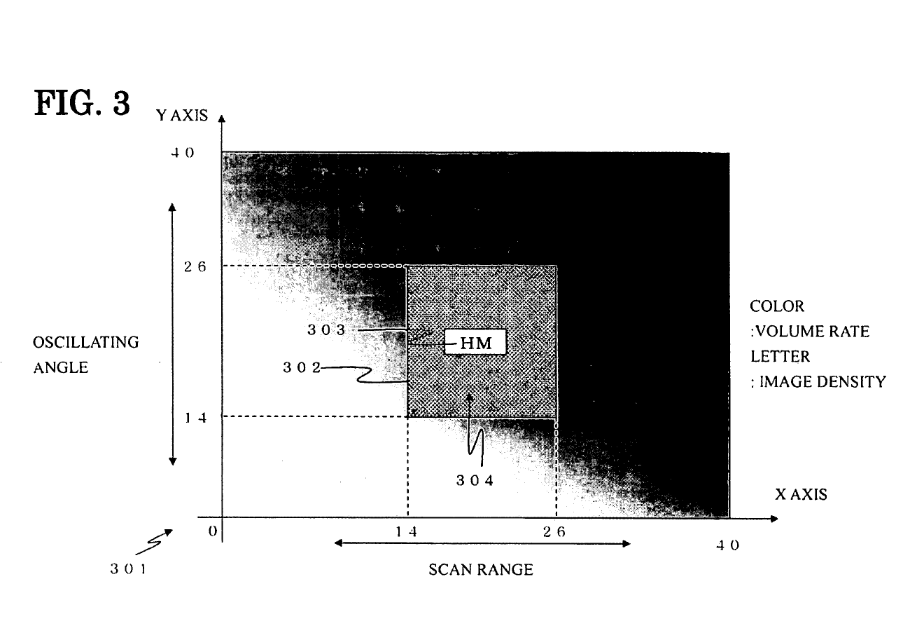

Next, we explain a case in which there are two parameters provided as fixed values and an instruction to alter the value for one of the remaining two parameters and the graphic-creating part 002 receives a newly determined value for the parameter through the parameter-calculator 003 based on the trade-off relationship. In this case, the graphic-creating part 002 creates the setting screen 301 as shown in FIG. 3 based on the new values for the parameters. FIG. 3 shows a setting screen of graphically displayed parameters altered by changing the parameters. For example, it is assumed that (w, x, y, z)=(w₁, x₂, y₁, z₂) is received as the new values for the parameters, x₂ is a value of the third level of the scan range and z₂ is a value of the fourth level of the volume rate. In this case, as shown in FIG. 3, the length in the X-axis direction that represents the scan range is determined to be 12 and the square 302 is altered. In addition, the color 304 that represents the volume rate is altered to be a gray. Furthermore, because the other two parameters are fixed values, the length in the Y axis and the letter 303 are not altered. This allows the user or operator to visually recognize the alteration of the value for the parameter in a trade-off relationship. In this case, as the scan range decreases, the length in the X-axis direction of the square 302 becomes shortened, while on the other hand, the volume rate increases. Therefore, as the color 304 becomes darker, the trade-off relationship between the scan range and the volume rate can be recognized visually.

In the present embodiment, to enhance the visibility, according to the alteration of the parameter assigned to the color, it uses gradation that represents a color for larger values as moving to the upper right in the background. The color display may be plain. In addition, to enhance the visibility, the letter may be described corresponding to the gradation. That is, in the present embodiment, an expression that refers to a larger value of the parameter assigned to the color, such as “High” or “Fast”, may be described in the upper right corner of the graphic, and an expression that refers to a small value of the parameter assigned to the color, such as “Low” or “Slow”, may be described in the lower left corner of the graphic.

The display controller 001 sends the values for the parameters that are entered and altered to the parameter-calculator 003 by using the user interface 100 having the display part 101 and the input part 102.

In the present embodiment, the user or operator directly enters the values for the parameters by using the input part 102. However, other methods may also be employed. For example, the operator who first use the input part 102 may allocate the four parameters to each expression method, and then the display controller 001 once sends the allocation to the graphic-creating part 002. The graphic-creating part 002 returns an appropriate graphic to the display controller 001. The display controller 001 instructs the display part 101 to display a tentative graphic. The display part 101 may be a touch panel. A method may be employed in which the user or operator touches the display part 101 to enter three of either the length in the X-axis direction, the length in the Y-axis direction, the letter, and the color. This can be done using the following method. Firstly, the length in the X-axis direction and the length in the Y-axis direction can be adjusted by holding the origin with one's fingers to extend to the opposite side relative to the central line of the axis. The graphic-creating part 002 receives the length via the display controller 001 and returns the corresponding value for the parameter to the display controller 001. In addition, the color can be adjusted by touching the gradation color in the background. The graphic-creating part 002 once receives the color via the display controller 001 and returns the corresponding value for the parameter to the display controller 001. Furthermore, by touching the letter with one's fingers, a list of letters sent from the graphic-creating part 002 is displayed. The graphic-creating part 002 once receives the letter selected from the list via the display controller 001 and returns the corresponding value for the parameter to the display controller 001. It allows the display controller 001 to obtain the value for the parameter for sending to the parameter-calculator 003.

The display controller 001 instructs the display part 101 to display the setting screen sent from the graphic-creating part 002.

When the user or operator enters a confirmation of parameters by using the user interface 100, the display controller 001 sends a command of parameter confirmation to the parameter-calculator 003.

When receiving the parameter confirmation and displaying the 3D dynamic image, firstly the display controller 001 deletes the setting screen used for the parameter setting. Next, the display controller 001 instructs the display part 101 to display the 3D dynamic image sent from the ultrasonic image generation part 005. In the present embodiment, the 3D dynamic image and the setting screen are displayed on the same place. They may be displayed on different places of the same display part 101 or in different display parts. In this case, the setting screen may remain displayed.

The ultrasonic image generator 005 is comprised by the ultrasonic probe 006, the transmitting and receiving circuit 007, and the image-processing circuit 008. Based on the values for the four parameters received from the parameter-calculator 003, the oscillating angle, scan range, image density, and volume rate are set. The ultrasonic image generator sends a signal from the transmitting and receiving circuit 007 based on the set image density and volume rate. The ultrasonic image generator 005 irradiates an ultrasound beam to the object by activating the ultrasonic probe 006 according to the set oscillating angle. In addition, according to the set scan range, the ultrasonic image generator 005 receives an ultrasonic echo from the subject that is received by the ultrasonic probe 006 in the transmitting and receiving circuit 007. Furthermore, the ultrasonic image generator 005 generates a 3D dynamic image in the image-processing circuit 008 based on the received ultrasonic echo. The ultrasonic image generator 005 then sends the generated 3D dynamic image to the display controller 001.

The execution controller 009 manages the overall performance of each functional part and information exchange in the above-mentioned parameter setting and 3D dynamic image creation.

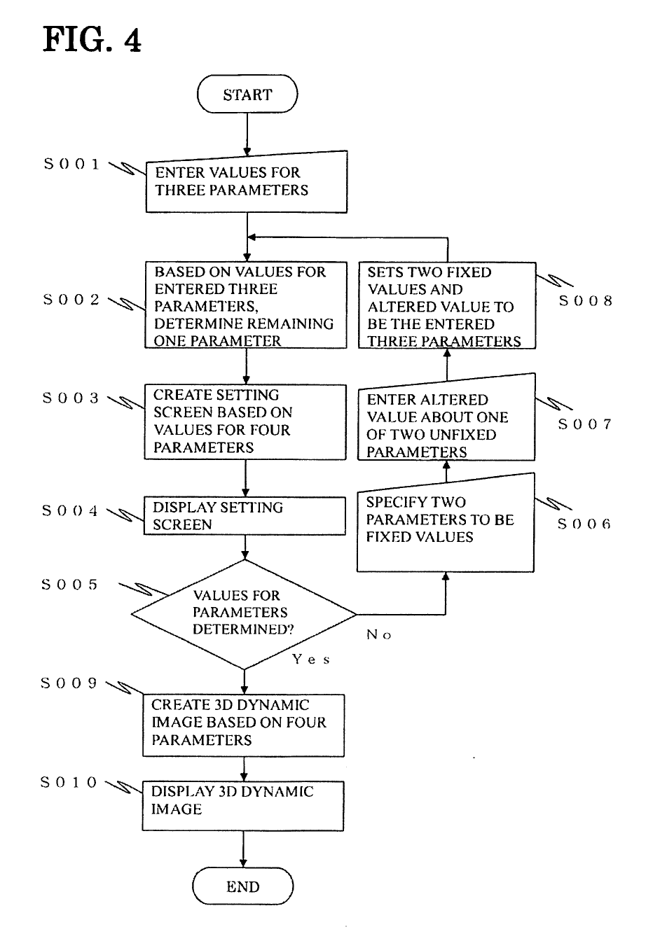

Next, with reference to FIG. 4, we explain the operational flow of parameter setting and the 3D dynamic image creation of the ultrasonic imaging apparatus according to the present embodiment. Herein, FIG. 4 is a flow chart of parameter setting and the 3D dynamic image creation of the ultrasonic imaging apparatus according to the present embodiment.

Step S001: The user or operator enters the values for three of the four parameters required for creating a 3D dynamic image by using the user interface 100.

Step S002: The parameter-calculator 003 receives the values for the three parameters from the display controller 001 and obtains the remaining one parameter by referring to the corresponding table of the memory 004.

Step S003: The graphic-creating part 002 creates a setting screen by using its own coordinate system based on the values for the four parameters received from the parameter-calculator 003. In addition, the graphic-creating part 002 determines the color and letter by using a corresponding table of its own parameters and colors or letters based on the allocation of each entered parameter to the respective display method.

Step S004: The display controller 001 instructs the display part 101 to display the setting screen received from the image-creating part 002.

Step S005: The user or operator refers to the displayed setting screen, determines whether or not to confirm the values for the parameters, and enters the confirmation if yes. If no, move on to Step S006. If yes, move on to Step S009.

Step S006: The user or operator specifies two parameters to be fixed values.

Step S007: The user or operator enters the altered value about one of the two unfixed parameters.

Step S008: The parameter-calculator 003 sets the two fixed values and the altered value to be the entered three parameters and returns to Step S002.

Step S009: The ultrasonic image-creating part 005 creates a 3D dynamic image based on the four parameters.

Step S010: The display controller 001 deletes the setting screen from the display part 101. Furthermore, the display controller 001 instructs the display part 101 to display the 3D dynamic image that has been entered from the ultrasonic image-creating part 005.

The ultrasonic imaging apparatus according to the present embodiment may be implemented by a program that specifies the parameter settings and the display action of the 3D dynamic image as mentioned above.

As described above, in the ultrasonic imaging apparatus according to the present embodiment, a setting screen is first displayed by using a correlating graphic, color, or letter that represents the values for two of four parameters required for generating a 3D dynamic image. Secondly, after altering the value for one parameter, based on the correlation of each parameter, it displays a setting screen with the altered graphic, color, or letter. It allows for displaying the setting conditions of the four parameters as one image. Because it is possible to visually recognize the setting conditions of the parameters, the values for four parameters can be set easily to their appropriate values. Accordingly, even users or operators (physicians) with little experience in creating a 3D dynamic image can create a 3D dynamic image that has the required size, image roughness, and smoothness of movement.

In the descriptions above, we have explained cases in which all four parameters are assigned to the correlating graphic, color, and letter that represent the values for two parameters and they are displayed. For this, it is not necessary to use all four parameters. For example, it may select two of the four parameters and use only the correlating graphic that represents the values for the parameters, so as to create a setting screen that represents the setting conditions of two parameters. It is also possible to select three of the four parameters and use a graphic that represents the color or letter and the value for the parameter, so as to create a setting screen that represents the setting conditions of three parameters.

### Second Embodiment

The ultrasonic diagnostic equipment according to the second embodiment is explained below. The ultrasonic diagnostic equipment according to the second embodiment is configured to determine the value for the parameter, instead of by referring to the table in the first embodiment, by utilizing a relational expression that represents the correlation of the four parameters. The method of obtaining the values for the parameters is explained below, the method utilizing the relational expression that represents the correlation of the four parameters. In the following description, w refers to the value of the irradiation range of the ultrasound beam in the slice direction (the “oscillating angle”), x refers to the value of the slice range, y refers to the image density, and z refers to the volume rate. Furthermore, in the following, we explain a case in which the volume rate is adjusted by entering each value of the oscillating angle, slice range, and image density.

The memory 004 stores in advance the relational expression that represents the correlations of the four parameters. Firstly, the number of scanning lines is represented by the oscillating angle, slice range, and image density; that is, F (w, x, y). The volume rate is inversely proportional to the number of scanning lines, and therefore represented by z=a/F (w, x, y) (a is a constant). Accordingly, in the present embodiment, z=a/F (w, x, y) is stored in the memory 004 as a relational expression.

The parameter-calculator 003 obtains the volume rate value z by assigning the entered values for three parameters (w, x, y)=(w₁, x₁, y₁) to the abovementioned expression. In this case, it is expressed as z=a/F (w₁, x₁, y₁).

Next, the parameter-calculator 003 receives from the display controller 001 an input of two parameters to be fixed values and an alteration of the value for one of the remaining two parameters. The parameter-calculator 003 assigns the two fixed values and the altered value to the abovementioned expression as the values for the three parameters and determines the value for the remaining one parameter. Herein, we explain a case in which the oscillating angle and the image density are the fixed values and the slice range value is altered to x₂. In this case, the parameter-calculator 003 takes (w, x, y)=(w₁, x₂, y₁) as the entered three values and assigns them to the above-mentioned relational expression to determine the volume rate value. Here, it is expressed as z=a/F (w₁, x₂, y₁).

The values for the four parameters determined as such are delivered to the graphic-creating part 002 as in the first embodiment. Based on these values, a setting screen is created. The display controller 001 instructs the display part 101 to display it. Furthermore, when the values for the parameters are confirmed, the ultrasonic image generator 005 creates a 3D dynamic image based on the values. The display controller 001 instructs the display part 101 to display the 3D dynamic image.

So far, we have explained a case in which the volume rate is determined by using the values for other parameters. However, the same applies to cases of determining any of the other values.

As mentioned above, in the present embodiment, because the value for the remaining one parameter is determined based on the values for three parameters by using a relational expression, the value for the parameter can be determined without levels. This allows for more detailed parameter setting to create a 3D dynamic image.

What is claimed is:

1. An ultrasonic imaging apparatus comprising:
a user interface having a display part and an input part to enter values for three among four parameters including an irradiation range of an ultrasound beam in a slice direction, an irradiation range of an ultrasound beam orthogonal to said slice direction, an image density, and the number of 3D image displays per unit time;
a parameter-calculator configured to calculate the value for the remaining parameter based on said values for three parameters and correlation information that represents the correlations between said four parameters stored in advance in a memory;
a graphic-creating part configured to execute: creating an image that represents the correlations graphically based on the values for two of said three entered parameters and said obtained parameter; creating a setting screen to instruct said display part to variably display both parameters and, in the case of receiving an instruction to alter the value for one of said two graphically expressed parameters, instructing said parameter-calculator to determine the other value based on said correlation information, so as to recreate said graphically expressed image;
a display controller configured to instruct the display part to display said setting screen; and
an ultrasonic image generator configured to generate a 3D dynamic image based on the data obtained by receiving an input to determine the value for said parameter, irradiating an ultrasound beam to an object based on said values for four parameters, and receiving an ultrasonic echo.

2. An ultrasonic imaging apparatus according to claim 1, wherein said correlation information is a corresponding table that represents the correlations using numerical values or a formula that represents the correlations.

3. An ultrasonic imaging apparatus according to claim 1, wherein said selected two parameters is variable at any time before or after creating said setting screen.

4. An ultrasonic imaging apparatus according to claim 1, wherein said graphic-creating part is configured to execute: representing by letter or color a value for one parameter among said entered three parameters and said obtained parameter; creating an image that represents said correlation graphically based on values for two of the other parameters; creating a setting screen to variably display said three parameters on said display part by combining said letter or color and said image, and in the case of selecting the displayed two of three parameters and instructing to alter the value for one of the selected two parameters by means of said input part, instructing said parameter-calculator to determine the other value based on said correlation information, so as to recreate said setting screen.

5. An ultrasonic imaging apparatus according to claim 1, wherein said graphic-creating part is configured to execute: representing by color a value for one parameter among said entered three parameters and said obtained parameter; representing by letter a value for one of the other three parameters; creating an image that represents the correlation graphically based on values for the remaining two parameters; creating a setting screen for said display part to variably display said four parameters by combining said color, said letter, and said image, and in the case of selecting two of four parameters and receiving an instruction to alter the value for one of the selected two parameters by means of said input part, instructing said parameter-calculator to determine the other value based on said correlation, so as to recreate said setting screen.

6. An ultrasonic imaging apparatus according to claim 1, wherein an image that represents correlation graphically based on said values for two parameters represents a value for one parameter by length in the lateral direction and a value for another parameter by length in the longitudinal direction.

7. An ultrasonic image acquisition method comprising;
calculating the value for the remaining parameter, from inputs of any values for three of four parameters, based on the correlation information on said four types of parameters stored in advance in the memory, the four parameters including: an irradiation range of an ultrasound beam in a slice direction, an irradiation range of an ultrasound beam orthogonal to said slice direction, an image density, and the number of 3D image displays per unit time;
creating an image that represents the correlation graphically based on values for two of said entered three parameters and said obtained parameter, to create a setting screen on which said display part variably displays the parameters;
displaying said setting screen on said display part;
repeating said calculation and said setting-screen-creation by defining said entered values for three parameters, as the values for the altered parameter and two non-graphically-represented parameters, when an instruction to alter the value for one of said two graphically represented parameters is received through the input part; and
irradiating an ultrasound beam to an object based on said entered values for three parameters and said obtained value for the remaining parameter when determination of values for each parameter are entered, so as to generate a 3D dynamic image based on the data obtained by receiving an ultrasonic echo from the object.

8. An ultrasonic image acquisition method according to claim 7, wherein said correlation information is a corresponding table that represents the correlations through numerical values or a formula that represents the correlations.

9. An ultrasonic image acquisition method according to claim 7, wherein said selected two parameters is variable at any time before or after creating said setting screen.

10. An ultrasonic image acquisition method according to claim 7, wherein said setting-screen-creation comprises: representing by letter or color a value for one of said entered three parameters and said obtained parameter; creating an image that represents the correlation graphically based on values for two parameters of the other parameters; and creating a setting screen that variably displays said three parameters on said display part by combining said letter or color and said image; and in the case of selecting two of said three displayed parameters and receiving an instruction to alter the value for one of the selected two parameters by said input part, said repeating comprises determining the value for the remaining parameter based on said correlation information, so as to recreate said setting screen.

11. An ultrasonic image acquisition method according to claim 7, wherein said setting-screen-creation comprises: representing by color a value for one parameter of said entered three parameters and said obtained parameter on said computer; representing by letter a value for one of the other three parameters; creating an image that represents the correlation graphically based on the values for the remaining two parameters; and creating a setting screen that variably displays said four parameters on said display part by combining said color, said letter, and said image; and in the case of selecting two of said four parameters and receiving an instruction to alter the values for one of the selected two parameters by said input part,
wherein said repeating comprises determining the remaining value based on said correlation, so as to recreate said setting screen.

12. An ultrasonic image acquisition method according to claim 7, wherein said setting-screen-creation comprises representing a value for one parameter by length in the lateral direction and a value for another parameter by length in the longitudinal direction, as an image that represents the correlation graphically based on the values for said two parameters.
